(19) United States
(12) Patent Application Publication
(10) Pub. No.: US 2002/0150627 A1
(43) Pub. Date: Oct. 17, 2002

(54) Composition containing creatine and phosphorus

(76) Inventors: Jeffrey Ray Stout, Boca Raton, FL (US); Rudolf Leonardus Lodewijk Smeets, Venlo (NL); George Verlaan, Wageningen (NL); Geessien Roelien Steenge, Arnhem (NL)

Correspondence Address: YOUNG & THOMPSON, 745 SOUTH 23RD STREET 2ND FLOOR, ARLINGTON, VA, 22202

(21) Appl. No.: 09/769,245

(22) Filed: Jan. 26, 2001

(51) Int. Cl. A61K 33/42; A61K 31/195; A61K 31/7004

(52) U.S. Cl. 424/601; 514/565; 514/23

(57) ABSTRACT

The invention discloses a composition comprising (a) soluble creatine, (b) a phosphorus supplement, wherein the phosphorus supplement provides at least 75% of the recommended daily dose of phosphorus value per serving, and (c) a blood buffer, which is preferably citrate and/or (bi)carbonate. The composition is used in a method for increasing the energy capacity within tissue cells.

### FIELD OF THE INVENTION

The invention pertains to nutritional compositions containing creatine and phosphors, and to methods of increasing the energy capacity and, in particular the anaerobic working capacity of humans and animals by administering such compositions.

### BACKGROUND OF THE INVENTION

The energy level of cells is dependent on numerous factors, often referred to as the cell metabolic state. Metabolism can be defined as the sum of all chemical reactions occurring in a cell, many of which are for breaking down nutrients, many of which are for building other molecules. Metabolism involves both catabolism and anabolism.

Catabolism is the decomposition of complex molecules to more simple molecules. It is performed by cell enzymes in order to extract covalent bond energy. The extracted energy is then used to perform anabolism, wherein energy derived from catabolic processes is converted to high energy phosphate bonds of for example adenosine triphosphate (ATP).

During high energy requiring activities, especially within muscle cells, the ATP pool is rapidly depleted and ATP has to be quickly resynthesized. It is believed that during highly intensive exercise for a limited period of time, ATP regeneration from ADP is for a significant part accomplished by the anaerobic degradation of phosphocreatine. Maximal anaerobic work is therefore believed to be limited by the breakdown of (ATP) and phosphocreatine (7). In line with this hypothesis, it has been demonstrated that high intensity exercise resulted in a 35 to 96% depletion of muscle phosphocreatine levels, depending upon the duration of the activity (13, 14, 19).

A number of studies have shown that anaerobic working capacity (AWC) from the critical power test provides a theoretically and experimentally valid estimate of work capacity associated with muscle energy reserves (4, 15, 16). The muscle energy reserves are formed by the ATP and phosphocreatine pools present wind the muscle cells. These reserves are especially important for subjects involved in intermittent and non-endurance activities, such as intermittent and non-endurance sports. Clear examples of such activities include for example athletics, field sports, cycling, running, bodybuilding, etc.

Creatine, the precursor of phosphocreatine, is an amino acid (N-amidino-N-methylglycine) that is found in many foods and feeds and is an important regulator in the energy supply and energy storage within the cell. Creatine is synthesized from glycine, arginine and methionine in the liver, pancreas and kidney and subsequently released into the blood stream. The cellular concentration of creatine is determined by the ability of the muscle cells to take up creatine because muscle cells cannot synthesize creatine. Phosphocreatine is generated by conversion of creatine to phosphocreatine by creatine kinase. The concentration of phosphocreatine in the muscle is believed to be about 4 times greater than ATP and is about 3 times greater than that of creatine.

Increased capacity of muscle cells to perform the energy consuming muscle contraction will increase performance. Therefore subjects involved in high energy consuming activities often are administrated nutritional supplements comprising compounds involved in cell metabolism, to enhance performance. For example, the (oral) administration of creatine-containing supplements is widely accepted by subjects involved in high energy consuming activities such as athletes and animals and is believed to improve performance by increasing intracellular muscle phosphocreatine/creatine pools.

A number of studies have demonstrated that a daily intake of creatine monohydrate 4×5 gram creatine per day, also referred to as creatine loading, for five to six days can significantly increase skeletal muscle creatine content by an average of 20%, with phosphocreatine accounting for 20% of the increase (12, 19). Additionally, several studies have demonstrated ergogenic benefits of creatine monohydrate loading on isokinetic strength (10), running (3), cycling (1, 2, 5, 11), jumping (3, 19) and bench-press (19) performance. Furthermore, Casey et al. (5) reported a significant correlation (r=0.72, p≦0.05) between anaerobic performance during isokinetic cycling and skeletal muscle creatine retention from creatine monohydrate loading.

WO 98/06278 describes methods and compositions for increasing the anaerobic working capacity in tissues and provides a method for increasing the synthesis and accumulation of beta-alanylhistidine dipeptides, with a simultaneous increase in the accumulation of creatine, in bodily tissues of humans and animals. This is accomplished by causing an increase in the blood plasma concentrations of beta-alanine and creatine, or the blood plasma concentrations of beta-alanine, L-histidine and creatine, by the ingestion or infusion of a composition including beta-alanine, beta-alanine and creatine, or beta-alanine, L-histidine and creatine, or active derivatives thereof.

WO 00/30634 relates to the use of creatine or a creatine analogue for reducing the biological process of aging, for treating disuse muscle atrophy or for stimulating subsequent restoration of muscle mass in rehabilitation training. The muscle atrophy is the result of immobilization, or reduced level of physical activity due to either disease or aging. The biological process of aging comprises for example the simultaneous degeneration and degradation of body tissues, including muscle and bone, neural tissue and secretory cells. The invention further relates to a therapeutic preparation for treating or preventing disuse muscle atrophy and for inhibiting the biological process of aging, comprising a suitable carrier, diluent or excipient and an effective amount of one or more creatine compounds

The exposure of creatine, such as creatine monohydrate, to the acidic environment of the stomach is believed result in an irreversible formation of creatinine from the creatine. Since creatinine has no function in the cell, creatinine is excreted.

U.S. Pat. No. 5,908,864 to provides a creatine supplement which does not quickly convert to creatinine and is easily and conveniently ingested by a person. Disclosed is a nutritional gel containing creatine and the method of producing the creatine gel. The creatine gel is made by cross linking maltodextrin and a modified starch through an aqueous endothermic reaction at a temperature of approximately 90 degrees Celsius. A buffering agent, such as potassium phosphate, is added to the gel to maintain a pH value at approximately 7.0. The gel is then cooled and creatine is added. Next, the gel is stabilized bacteriologically by adding a preservative, such as potassium sorbate to the gel.

U.S. Pat. No. 5,973,199 provides hydrosoluble organic salts of creatine. The salts are useful in the dietetic and food industry.

U.S. Pat. No. 5,925,378 provides a method for enhancing a stable concentration of cellular creatine in a human includes dissolving an effervescent containing an acidic edible salt form of creatine in water. Once the mixture has completely dissolved the solution is immediately ingested, and an effective amount of creatine is absorbed. Preferably, the effervescent is in the form of a tablet which contains creatine m the form of an edible salt, a mixture of acids, and sodium.

Besides creatine, phosphate and other phosphorus compounds play an important role in energetic capacity within the body. Phosphate plays a vital role in ATP resynthesis and phosphocreatine synthesis, since it is a substrate in the phosphorylation of ADP and creatine. Inorganic phosphate (Pi) supplementation is believed to provide a number of ergogenic benefits. It has been reported to increase aerobic capacity by increasing 2,3-diphospho-glycerate (DPG) levels in red blood cells (Cade et al., Med Sci Sports Exerc 16(3):263-8, 1984; Kredier et al., Med Sci Sports Exerc 22(2);250-6, 1990). The principle role of 2,3-DPG is to facilitate oxygen release from the binding of haemoglobin so that the oxygen can diffuse faster into working muscle cells.

Several other mechanisms have been proposed through which phosphate supplementation may affect performance, besides increase of 2,3-DPG levels. These include the promotion of phosphate-stimulated glycolysis, increased availability of phosphate for oxidative phosphorylation and creatine phosphate synthesis, an increased red cell anaerobic glycolytic efficiency, and enhanced myocardial and cardiovascular efficiency. Although theoretical rationales for phosphate supplementation for ergogenic benefits are known in the art, the ergogenic benefits of phosphate supplementation have not been consistently observed and little scientific evidence exists to recommend exogenous phosphate as an ergogenic aid (21). Concurrent administration of phosphate and creatine has been suggested in the art to have additional effects.

U.S. Pat. No. 5,968,544 discloses an acidic composition for human consumption, comprising creatine. In particular the composition is conveniently an isotonic drink for storage at 4° C. or is a dry stable powder which may be stored at ambient temperature. Optionally included in the preparation described are minerals in is a limited quantity, e,g. typical amounts of phosphates included are 50 mg/liter.

Recently, Wallace et al. (20) studied the effect of tie separate and combined short term creatine monohydrate and sodium phosphate supplementation on the body composition, performance, and blood chemistry. Subjects were randomly assigned to ingest either 1000 mg of tribasic sodium phosphate (P), 5000 mg creatine monohydrate (CR), 5000 mg creatine monohydrate and 1000 mg tribasic sodium phosphate, or a glucose placebo (G) four times daily for 5 days. Results indicated that following the phosphate trails VO2 max (8%) and the ventilatory anaerobic threshold were significantly elevated (10%). In contrast, both creatine monohydrate loading trails resulted in a significant increase in body mass (1.35±0.22 kg, mean SE) with no change in fat free mass (p<0.05). The only significant increase observed for concurrent creatine and phosphate loading compared to individual phosphate or creatine loading was an elevated power output suggesting that there may be some performance benefit to short-term creatine and phosphate loading. However, the effect of concurrent creatine and phosphate loading (6% increase in power output compared to placebo) is a mere additional effect of the phosphate (3% increase in power output compared to placebo) and creatine loading (3% increase in power output compared to placebo).

U.S. Pat. No. 5,973,005 provides a stable aqueous solution of creatine acid sulfate provides a source of creatine to an animal when taken orally. The aqueous solution of creatine acid sulfate (after neutralization and buffering) has a pH of about 7.2 to about 7.8 and is stable for at least six months at room temperature. The creatine acid sulfate is produced by adding creatine monohydrate to a sulfuric acid solution in a stoichiometric amount to result in creatine acid sulfate having a pH initially of 2.0-3.0. The resulting creatine acid sulfate is diluted with water and neutralized to raise the pH and avoid the formation of creatinine. The resulting creatine acid sulfate solution preferably contains a buffering and neutralizing agent such as tribasic potassium phosphate which forms mono- and dibasic potassium phosphates by interaction with the hydrogen ions liberated from the acid sulfate. The aqueous solution can be combined with a sweetener, electrolyte and carbohydrate source to produce a stable drink for providing a source of creatine to an animal in need thereof. An effective amount of glycerol is preferably added to enhance absorption of the creatine through the intestinal wall into he bloodstream and eventually to the needy skeletal muscles.

U.S. Pat. No. 5,397,786 discloses a liquid composition to be used as a rehydration drink, particularly suited for the administration to people who do heavy work under severe conditions, e.g. at high temperatures, and to sports people and athletes, as well as to patients who exhibit dehydration symptoms due to severe illnesses such as diarrhea or vomiting, contains per serving unit water at least 1 to 100 g of at least one carbohydrate, such as glucose polymers, maltodextrin and fructose; 2 to 2500 mg of at least one electrolyte, such as an alkali and/or earth alkali salt; 0, 1 to 750 mg of at least one ammonia neutralizer, such as D, L-magnesium aspartate, L-arginine and glutamate; at least one energy enhancer, such as members of the vitamin B group and branched chain amino acids; at least one antioxidant such as beta-carotene, vitamin C, vitamin E and selenium; 1 to 30 mg of at least one membrane stabilizer, such as choline chloride, betaine chloride and methionine; and 1 to 200 μg of at least one neuromuscular function enhancer such as octacosanol.

During high intensity exercises the anaerobic conversion of glucose, i.e. glycolysis, results in the formation of hydrogen ions (H⁺), inducing chemical changes, e.g. a decrease of blood pH. Decrease of blood pH results in for example pain and leads to decreased performance of muscles. Blood buffers in the blood protect against large changes in pH. It is believed within the art that suitable buffering substances, when taken orally, can increase performance.

The energy requirement for the covalent bonding of phosphate to ADP is very often generated in the Krebs cycle. The Krebs cycle involves a series of enzymatically controlled reactions enabling complete oxidation of two-carbon acetyl groups to water and carbon dioxide. Each complete turn of the Krebs cycle yields one molecule of ATP, 3 molecules of NADH and one molecule of FADH2. The later are subsequently used as electron donors in the electron transport system to yield additional ATP.

WO 99/21565 describes a pharmaceutical composition which includes a sugar and a Krebs cycle intermediate, or salt thereof, or a precursor of a Krebs cycle intermediate. Krebs cycle intermediates include citric acid, aconitic acid, isocitric acid, alpha-ketoglutaric, succinic acid, fumaric acid, malic acid, and oxaloacetic acid, and mixtures thereof. Precursors of Krebs cycle intermediates are compounds converted by the body to form a Krebs cycle intermediate. The pharmaceutical composition is described to be particularly desirable for the prophylaxis or treatment of disorders associated with impaired mitochondrial function. Disorders that can be treated include conditions or diseases characterized by a decreased level of oxidative metabolism, such as conditions or diseases of the nervous system, conditions or diseases of other parts of the body and conditions or diseases of the body as a whole. The pharmaceutical composition described can also include an adjuvant for enhancing mitochondrial function (i.e., oxidative metabolism). Suitable adjuvant include vitamins, minerals, antioxidants, and other metabolism-enhancing compounds. Exemplary metabolism enhancing compounds include L-carnitine and its derivatives, and creatine.

Notwithstanding the above disclosures, there remains a need in the art for compositions, which increase the capacity of energy output from cells. Especially, compositions suitable for enhancing body strength or performance in intermittent and non-endurance sports, for example prevention, extension and treatment of muscle fatigue especially during high energy requiring activities for a relatively short period of time are desired. The composition thus solves problems with respect to insufficient power output, especially insufficient short-term power output. The composition according to the invention increases the power output, especially increases anaerobic working capacity of subjects in need thereof.

### SUMMARY OF THE INVENTION

The present invention provides desired and novel compositions, which increase the capacity of energy output from cells especially, provides for a composition for optimizing performance, especially muscle performance and/or enhancing muscle cell recovery after exercise.

The invention provides a composition, which shows surprisingly synergistic effects of creatine, phosphorus and blood buffers. Without being bound by theory, it is the inventors' believe that this synergistic effect is caused by the simultaneous increase of cellular phosphocreatine and ATP pool, increase of glycolytic capacity and counteracting the adverse side effects of increased glycolytic capacity. These concurrent actions result in a significant increase in anaerobic working capacity, providing cells, especially muscle cells, the opportunity to increase energy output and thus the ability to perform muscle contraction.

The invention further pertains to compositions for increasing the aerobic working capacity of a subject, making the composition suitable for subjects in need of increased performance, for example by remaining performance at a high level for a longer period of time.

### DETAILED DESCRIPTION OF TEE INVENTION

The composition according to the invention is especially suitable for the subjects in need of an increased energy capacity wit tissue cells, for example an increased power output and/or high power output for a longer period of time. Subjects wishing or requiring enhanced performance of the body such as an increased capacity to perform muscle contractions, or increased power output for a relatively short period of time can use the composition according to the invention advantageously. Furthermore, the composition according to the invention can be used in a method for treatment or prevention of disorders, diseases or abnormal physical states, especially associated with energy levels within the cell.

The composition was shown to be especially suitable for increasing the anaerobic working capacity (AWC) of a subject. AWC can be defined as the capacity to extract energy from phosphocreatine/ATP pools and/or glycolysis. Glycolysis is defined as the ATP generation via conversion of glucose or glycogen to lactate. A enhancement of the AWC accomplished by the composition according to the invention will for example result in: a) the ability to increase “explosive” power output, i.e. exercise wherein energy is mainly extracted from glycolysis and ATP/phosphocreatine pool; b) an extended exercise length; c) increased performance during competition; d) increased ability to recover from high intensity activities or e) increased performance in other activities with high energy requirements over a relatively short time period. Subjects wishing or requiring an increased AWC are often preparing for or practicing intermittent and non-endurance sports.

It was unexpected that the AWC would increase by administration of the composition according to the invention, although it has been suggested in the art that phosphate supplementation could increase the effects of creatine loading. AWC increase has been reported as an effect of creatine administration, however, phosphorus supplementation is believed in the art to increase aerobic capacity, via stimulation of 2-DPG formation. Results according to Wallace et al indeed suggest that the effect of concurrent phosphate and creatine supplementation on power output is merely based on an additional effect and not a synergistic effect. Table 1 shows that the concurrent administration of creatine and phosphate has a mere additional effect on the power output (results from Wallace et al).

Furthermore, it was shown that the effect on AWC achieved with a composition according to this invention is not a mere additional effect of phosphate, creatine and blood buffer supplementation, since the combination creatine/blood buffer only caused a 16% increase in anaerobic working capacity, while with composition according invention an increase of about 50% was observed. Although not wishing to be bound by theory, the inventors believe that the increased effect is caused by the combination of phosphorus, creatine and blood buffer, wherein creatine and phosphorus provide substrates for the intracellular creatine/phosphocreatine pool, wherein the phosphorus supplement increases glycolysis and wherein the blood buffer counteracts the effect of an increased glycolysis caused by the phosphorus supplementation. As a direct consequence of reduced adverse effects of increased glycolysis, glycolysis reactions and other reactions required for muscle contractions will proceed more efficiently and/or for a longer period of time, compared to a situation wherein the adverse effects of increased glycolysis are not counteracted. This results in an increased ability of cells to provide energy.

Table 1: Increase power output and anaerobic working capacity compared to placebo

TABLE 1
Increase power output and anaerobic
working capacity compared to placebo

| | Power output | |
|---|---|---|
| | according to | AWC using composition |
| | Wallace et al | of the invention |
| Placebo | — | — |
| Creatine | 3% | nd |
| Phosphate | 3% | nd |
| Creatine + Blood buffer | nd | 15.6% |
| Creatine + Phosphate | 6% | nd |
| Creatine + Phosphate + | nd | 49.8% |
| Blood Buffer | | |

The composition according to the invention is also especially useful for subjects which experience or need to prevent a decrease or insufficient increase in power output and/or anaerobic working capacity. The composition according to the invention can thus also be advantageously used prior to, dug or after for example muscle disuse, e.g. due to partial or complete immobilized of the body, e.g. due to hospitalizing, subjects spending a significant time in an environment with reduced gravitation such as astronauts; etc. Use of the composition in such events will prevent a large drop-back of muscle quality and facilitate recovery.

The creatine used in the composition according to the invention can be any creatine known in the art including for example creatine monohydrate, creatine phosphate, creatine sulfate. Preferably, however, creatine comprises a creatine salt consisting of anionic and cationic part, wherein the cationic part consists of and provides creatine or creatine analogs or derivatives.

Preferably a highly hydrosoluble salt of creatine is used as a source of creatine, since it is believed that a high solubility of creatine facilitates the absorption of creatine by the intestines and ultimately results in significantly higher increase of the intracellular creatine and phosphocreatine concentrations. Preferred hydrosoluble creatine salts are those having a solubility above about 6 grams of the salt per 100 ml water of 37° C., preferably above about 7.5 grams of the salt per 100 ml water, even more preferably above about 10 grams of the salt per 100 ml water of about 37° C. The solubility of the creatine salt used in the composition according to the invention is preferably further characterized by a limited time required to dissolve the organic salt of creatine.

The creatine salt to be used is especially a highly soluble organic creatine salt. Organic creatine salts are those creatine salts wherein both the anionic and cationic part of the salt are organic. The organic anionic part or the organic creatine salt can serve important functions within the subjects body, without the need for addition of a specific additional component or composition, which can make the composition unnecessarily complex and/or expensive. Preferred anions are those derived from (aliphatic) polycarboxylic, especially dicarboxylic and tricarboxylic organic acids, preferably having 3-6 carbon atoms, optionally containing 1-2 carbon-carbon double bonds, 1-4 hydroxyl groups and/or 1-2 keto groups, including citrate, maleate, tartrate and maleate. According to a particularly preferred embodiment, the anionic part of the creatine salts is capable of providing a blood buffer, for example citrate, or a Krebs cycle intermediate or precursor thereof, for example maleate, fumarate or citrate. Especially preferred creatine salts include those wherein the anion provides both a blood buffer and a Krebs cycle intermediate, for example creatine citrate. According to a preferred embodiment, a creatine salt is used having citrate as the anionic part and protonated creatine as the cationic part of the salts. Both creatine citrate and dicreatine citrate are suitable, and wherever creatine citrate is used in this disclosure, this can be replaced by dicreatine citrate and vice versa.

The quantity of creatine provided per weight of creatine salt depends on the type of creatine used. Preferably the composition according to the invention provides about 0.01 to about 1, preferably about 0.02 to about 0.5, most preferred about 0.04 to about 0.25 grams creatine per kilogram body weight per serving. For human beings the composition would provide about 0.5-100 grams creatine per serving, more preferably about 1-50 grams, most preferred about 2-25 grams, for example about 2.5-10 gram.

Without being bound by theory, the inventors believe that the phosphorus supplement within the composition according to the invention increases the ability of cells to provide energy via the glycolysis. During glycolysis, glucose or glycogen is first converted to glucose-6-phosphate, requiring phosphorus, and ultimately to pyruvate. Pyruvate can serve as a substrate within the Krebs cycle (aerobic) or results in the formation of lactate and hydrogen ions. The term glycolysis whenever used in the context of this disclosure refers to the conversion of glucose into lactate and hydrogen ions. Additionally, the phosphorus may serve as a substrate for the formation of ATP/phosphocreatine pool.

The recommended daily intake of phosphorus for humans differs in each country and is depending on age and sexes. According to the Nordic Nutrition Recommendations of 1996 (well known to those skilled in the art), about 700 mg phosphorus should be ingested by a young human on a daily basis and about 600 mg phosphorus in recommended for humans which have an age above 22 years. However, the US recommended daily allowances (RDA) is 700 mg for the group above 22 years. For clarity, whenever referred to a daily dope value for humans in the disclosure of this invention, this daily dose will refer to 700 mg phosphorus for a human being. The 700 mg daily dose within this invention is merely used as a constant for clarity, since the recommendations vary significantly per country, age, etc. The use of this constant therefore does not limit the scope of this invention, if alternative values for recommended doses are used. Mere recalculation based on the weight of phosphorus used in the composition according to the invention will provide the percentage of phosphorus based on the alternative recommended daily intake for phosphorus. For non-human mammals, the amounts to be used can be calculated in an analogous manner, on the basis of the recommended or usual daily intakes. The term phosphorus supplement includes any supplement, organic or inorganic, wherein a significant amount of phosphorus is present. Preferably the phosphorus is not part of the creatine (salt). Where amounts of phosphorus are given, these refer to elemental phosphorus (P).

The phosphorus supplement included in the composition according to the invention should provide in at least about 75% of the daily dose value for phosphorus per serving, preferably above about 90%, even more preferred above about 100%, especially preferred above about 110%, for example above about 125%. The composition should not exceed about 25 times the daily dose value of phosphorus per serving, preferably it comprises below about 15 times the daily dose value. For humans a daily dose of the composition would thus preferably provide above about 500 mg phosphorus per serving, preferably above about 600 mg phosphorus, especially preferred above about 700 mg phosphorus, for example 1000 mg phosphorus, however should not exceed 17.5 gram, preferably should be below about 10 grams.

Preferably the composition according to the invention has a weight ratio of phosphorus:creatine of about 1:25 to about 10:1, preferably about 1:10 to about 1:1, most preferably about 1:6 to about 1:4. Especially preferred are those composition providing both at least about 75% of the recommended daily dose of phosphorus and have a phosphorus to creatine ratio of about 1:25 to about 10:1.

Suitable phosphorus containing supplements include those supplements comprising bio-available phosphorus, for example salts including phosphate. According to a preferred embodiment, inorganic salts including phosphates, for example sodium phosphate, potassium phosphate are used. According to a preferred embodiment a mixture comprising sodium phosphate and potassium phosphate is used, wherein the potassium: sodium ratio is between 5:1 and 1:5 on a molar basis. According to a further preferred embodiment, mono-basic phosphate salts are used as a source of phosphorus.

Without being bound by theory, the inventors believe that the blood buffer is required in the composition according to the invention since it inhibits the undesired side effects of increased glycolysis, such as blood pH decrease. The term blood buffer includes all such compositions recognized in the art as suitable blood buffers. Examples of such buffers include salts comprising carbonate, such as sodium bicarbonate, potassium bicarbonate, sodium carbonate, potassium carbonate or mixtures including one of these compounds or citrate or citric acid. Unsuitable for use as a blood buffer is phosphate, since phosphate buffers fluids within the body such as urine. Furthermore sulfate is considered as unsuitable, as it provides insufficient, if any, blood buffering capacity and the smell and taste of sulfate are not appealing.

Since a high intake of vast amounts of carbonate has several negative side effects, such as explosive diarrhea, according to a preferred embodiment, the blood buffer comprises only a limited amount of carbonate or bicarbonate, in combination with a second suitable blood buffer, for example citrate and/or citric acid. According to a preferred embodiment, the composition according to the invention comprises about a weight ratio buffer:creatine of about 1:50 to about 50:1, preferably 1:10 to about 10:1, even more preferred 5:1 to about 1:5. Suitable amounts of carbonate and/or bicarbonate include above about 10 mg per gram of creatine, preferably above about 100 mg per gram of creatine, more preferred above about 250 mg per g creatine. Suitable amounts of citric acid include above about 10 mg per gram of creatine, preferably above about 100 mg per gram of creatine, more preferred above about 250 mg per gram creatine. Suitable amounts of citrate are above about 10 mg per gram of creatine, preferably above about 100 mg per gram of creatine, more preferred above about 200 mg per gram creatine.

According to a further preferred embodiment the composition according to the invention comprises one or more Krebs cycle intermediates or precursors thereof. Krebs cycle intermediates are the acids, which are utilized in or during the Krebs cycle. Thus, Krebs cycle intermediates include citric acid, aconitic acid, isocitric acid, succinic acid, fumaric acid, malic acid oxaloacetic acid. Precursors of Krebs cycle intermediates are those which upon administration to a subject are convened by the body into a Krebs cycle intermediate. Precursors of Krebs cycle intermediates include mono- and di-alkyl citrates, aconitates, isocitrates, α-ketoglutarates, succinates, fumarates, malates, and oxaloacetates and are especially suitable. Preferably citrates, fumarates, malates or mixtures including one of the previous are included. Suitable sources for Krebs cycle intermediates or precursors thereof include salts including precursors of Krebs cycle intermediates, for example sodium citrate or Krebs cycle intermediates such as for example citric acid. According to a particularly preferred embodiment the Krebs cycle precursor is provided by the anionic part of the creatine salt.

The composition according to the invention advantageously further comprises adjuvant for enhancing mitochondrial function, e.g. the oxidative metabolism. Suitable adjuvant include vitamins and minerals, for example vitamin B, especially vitamin B6, vitamin B1 and/or vitamin B2. Composition according to the invention can also include an adjuvant for enhancing the cells metabolism. Such compounds include L-carnitine and carbohydrates.

Carbohydrates included within the composition according to the invention can be any carbohydrate known in the art, suitable and able to ultimately function as a Krebs cycle substrate, or a pentose such as ribose. Such carbohydrates thus include digestible mono-saccharides and polysaccharides. Especially preferred monosaccharides include glucose and fructose. Preferred polysaccharides include sucrose, lactose, maltose, starch and starch fractions. Non-digestible carbohydrates (nutritional fibres) may also be present

The quantity of digestible carbohydrates included in the composition of the invention greatly depends on the purpose and subject to which the composition according to the invention is administrated. For example, the composition can be admixed to feed or food having a significant carbohydrate content. For use by humans, the composition of the invention preferably provides about 0.5 to about 500 grams of carbohydrates, more preferably about 1 to 200 grams, most preferably about 5-100 grams on a daily basis, for example 5-15, especially about 12 grams per serving.

The composition according to the invention further advantageously comprises compound or mixture of compounds having an effervescent action. Creatine may thereby be uniformly and accurately dispensed when completely dissolved in liquid. Suitable compounds include sodium bicarbonate, potassium bicarbonate, sodium carbonate, potassium carbonate or mixtures including one of these compounds. The inclusion of an effervescent will facilitate ease of usage of the composition, since it will decrease preparation time and facilitate proper mixing of the composition according to the invention with water. According to a preferred embodiment, the composition used as an effervescent additionally has the capacity to provide blood buffering capacity.

The composition according to the invention can further comprise compositions suitable for providing taste or color. Exemplary taste modifiers include artificial or natural flavorings such as citric flavors, e.g. fruit punch having lemon, orange or grapefruit and artificial sweeteners such as sucralose, aspartame or saccharin. Coloring can be provided by e.g. beta-carotene.

An especially effective method for increasing the anaerobic working capacity comprises a building phase and a maintenance phase. During the building phase the composition according to the invention is administrated 1.5-10 times the quantity required in the maintenance phase, preferably about 2.5-6 times. The building phase generally requires about 2-15 days for a human, for example about 4-9 days. Preferably about 4 servings per day are administrated a subject during the building phase and about 1 per serving is administrated to a subject during the maintenance phase. Alternatively, servings with about 4 times lowered levels of the active components can be served during the maintenance phase. For optimum results the subject is subjected to physical exercise during or after the building phase and during the maintenance phase.

The composition according to the invention can be advantageously used by human subjects. Especially human males can use the composition advantageously. Although human female subjects showed a 10.8% increase in AWC after 6 days supplementation compared to placebo, human males showed a 49.8% increase in AWC after 6 days supplementation compared to placebo.

The composition according to the invention is preferably administered orally as a nutritional supplement. The composition can for example be added to food or feed products. Preferably the composition is provided in an edible bar or cookie, a soluble powder, drink, tablets or capsule.

### EXAMPLE 1

### Sports Trial

Thirty-one healthy men were tested.

Following pre-testing, the subjects were randomly assigned to one of three treatment conditions using a double blind design:

1) 20 g of flavored dextrose (CHO) plus blood buffer in the form of effervescent carbonate/bicarbonate powder as a placebo (PL);

2) 18 g CHO, blood buffer and 5 g creatine, the blood buffer and the creatine being provided in the form of creatine citrate, and effervescent carbonate/bicarbonate powder in a flavored effervescent powder blend (Creatine Edge Effervescent; Fortress Systems, Omaha) (Cr);

3) 18 g CHO, blood buffer, 5 g creatine and 1 g phosphorus, the blood buffer and the creatine being provided in the form of creatine citrate, and effervescent carbonate-/bicarbonate powder in a flavored effervescent powder blend (Creatine Edge Effervescent; Fortress Systems, Omaha) (CP).

The subjects ingested the supplements four times per day for two and six consecutive days before returning to the lab for post-testing.

Critical Power Test

Thirty healthy men completed three phases of testing on a calibrated electronically braked cycle ergometer (Corival 400, Quinton Instruments).

1) pre-testing did consist of two bouts (=exercise session) performed at power outputs selected to elicit fatigue in 1 to 10 minutes;

2) post-testing following two days of supplementation required two work bouts at power outputs identical to those performed during pre-testing.

3) post-testing following six days of supplementation required two work bouts at power outputs identical to those performed during pre-testing.

The procedure for the critical power (CP) test is the same as previously described by Moritani et al. (16), The power outputs for the exercise bouts ranged from 200-375 watts (W) depending upon the fitness level of the subject. Rest periods between each exercise bout was continued until the subjects' heart rate returned to within 10 beats per minute of pre-exercise levels (this usually takes 30 minutes or longer; 20-21). The subjects performed both exercise bouts on one day.

Prior to each exercise bout, the seat height of the cycle ergometer was adjusted for near full extension of the subject's legs while pedaling and foot straps were adjusted to prevent the feet from slipping off the pedals during testing. Subject did warm up for four minutes by pedaling at a power output of 30 W. Following a 2-minute rest period, the subjects began pedaling against zero resistance and, upon reaching a pedaling rate of 70 rpm, the appropriate power output was applied within the first 2-3 seconds of the test. Each subject was encouraged to maintain the required pedaling rate throughout the entire exercise bout. The exercise bout was immediately terminated when the subject was unable to maintain 65 rpm as determined by the rpm monitor on the ergometer. Time limit (TL) was recorded to the nearest 0.1 second.

Work limit (WL) was calculated by multiplying power (P) and TL (WL=P×TL), Anaerobic working capacity (AWC) is the amount of work in kilojoules corresponding to the Y intercept of the WL-TL relationship as previously described (15-16). Test-retest reliability data for AWC (anaerobic working capacity) for young adult males (n=11) measured 7 days apart resulted in an intraclass correlation (R) of 0.97 with a SEM of 0.59 kJ.

Results

Table 2: Mean AWC (kJ) for each treatment group before treatment (0 days) after 2 days and after 6 days of supplementation. % change represents the average increase in mean AWC after 6 days supplementation.

TABLE 2

| Group | n | 0 days | 2 days | 6 days | % change |
|---|---|---|---|---|---|
| Blood buffer | 10 | 16.2 | 13.6 | 15.7 | 0% |
| Creatine + buffer | 10 | 17.5 | 16.7 | 20.2 | 15.6% |
| Creatine + buffer + P | 11 | 15.0 | 18.5 | 22.4 | 49.8% |

It was surprisingly found that the composition according to the invention resulted in a much greater increase in AWC than the ingestion of creatine without phosphate.

### EXAMPLE 2

### Soluble Powder

A powder to be reconstituted in water or fruit juice comprising,

7.7 grams dicreatine citrate (provides 5 gram creatine)

12 grams fructose

2.06 grams monobasic potassium phosphate

2.06 grams monobasic sodium phosphate

3.8 grams sodium bicarbonate

150 mg sodium carbonate

4.6 grams citric acid

artificial/natural fruit punch

artificial colors

### EXAMPLE 3

### Solubility

Using HPLC, the saturated solubility of dicreatine citrate and creatine monohydrate in water of 37° C. was determined to be 148.1 (±0.7) and 57.6 (±9.3), respectively. Thus the saturated solubility of dicreatine citrate is about 2.6 times higher than that of creatine monohydrate.

References

1. Balsom, P. D., B. Ekblom, K. Soderlund, B. Sjodin, E. Hultman. Creatine supplementation and dynamic high-intensity intermittent exercise. Scand. J. Med. Sci. Sports. 3:143-149, 1993.

2. Birch, R., D. Noble, and P. L. Greenhaff. The influence of dietary creatine supplementation on performance during repeated bouts of maximal isokinetic cycling in man, Eur. J. Appl. Physiol. 69:268-270, 1994.

3. Bosco, C., J. Tihanyi, J. Puospk, I. Kovacs, A. Gabossy, R. Colli, G. Pulvirenti, C. Tranquili, C. Foti, M. Viru, and A. Viru. Effect of oral creatine supplementation on jumping and running performance. Int. J. Sports Med. 18:369-372, 1997.

4. Bulbulian, R., J. W. Jeong, and M. Murphy. Comparison of anaerobic components of the Wingate and Critical Power tests in males and females. Med. Sci. Sports Exerc. 28,1336-1341, 1996.

5. Casey, A., D. Constantin-Teodosiu, S. Howell, E. Hultman, and P. L. Greenhaff. Creatine ingestion favorably affects performance and muscle metabolism during maximal exercise in humans. Am. J. Physiol. 271 :E31-E37, 1996.

6. Cook, W. H., P. W. Grandjean, and W. S. Barnes. Effect of oral creatine supplementation on power output and fatigue during bicycle ergometry. J. Appl. Physiol. 78:670-673, 1995.

7. deVries, H. A., and T. J. Housh. Physiology of exercise: for physical education, athletics and exercise science. 5^{th} Ed, Brown and Benchmark. Madison, Wis., pp 37-39, 1994.

8. Green, A. L., E. J. Simpson, J. J. Littlewood, I. A. Macdonald and P. L. Greenhaff. Carbohydrate ingestion auments creatine retention during creatine feeding in humans. Acta Physiol. Scand. 158:195-202, 1996.

9. Greenhaff, P. L., K. Bodin, K. Soderlund, and E. Hultman. The effect of oral creatine supplementation on skeletal muscle phosphocreatine resynthesis Am. J. Physiol. 266:E725-E730, 1994.

10. Greenhaff, P. L., A. Casey, A. H. Short R. Harris, K. Soderlund, and E. Hultman. Influence of oral creatine supplementation on muscle torque during repeated bouts of maximal voluntary exercise in man. Clin. Sci. 84:565-571, 1993.

11. Hall, E. L., J. C. Smith, D. P. Stephens, P. G. Snell, and C. P. Earnest. Effect of oral ingestion of creatine monohydrate on parameters of the work-time relationship. Med. Sci. Sports Exercise. 25(Supplement):S15, 1995.

12. Harris, R. C., K. Soderlund, and E. Hultman. Elevation of creatine in resting and exercised muscle of normal subjects by creatine supplementation. Clin. Sci. 83:367-374, 1992.

13. Hultman, E., J. Bergstrom, and N. McLennan-Anderson. Breakdown and resynthesis of phosporylcreatine and adenosine triphosphate in connection with muscular work in man. Scand. J. Clin. Lab. Invest. 19:56-66, 1967.

14. Karlsson, J., B. Diamant, B. Saltin. Muscle metabolites during submaximal and maximal exercise in man. Scand. J. Clin. Lab. Invest. 26:385-394, 1971.

15. Monod, H., and J. Scherrer. The work capacity of a synergic muscular group. Ergonimics. 8;329-338, 1965. Moritani, T., A. Nagata, H. deVries, and M. Muro. Critical power as a measure of physical work capacity and anaerobic threshold. Ergonomics, 24:339-350, 1981.

16. Nebelsick-Gullett, L. J., T. J. Housh, G. O. Johnson, and S. M. Bauge. A comparison between methods of measuring anaerobic work capacity. Ergonomics 31: 1413-1419, 1988.

17. Odland, L. M., S. D. MacDougall, M. A. Tarnopolsky, A. Elorriaga, and A. Borgmann. Effect of oral creatine supplementation on muscle [PCr] and short-term maximum power output. Med. Sci. Sports Exerc. 29:216 219, 1997.

18. Volek, J. S., W. J. Kraemer, J. A. Bush, M. Boetes, T. Incledon, K. L. Clark, and J. M. Lynch. Creatine supplementation enhances muscular performance during high-intensity resistance exercise. J. Am Diet. Assoc. 97:765-770, 1997.

19. Volek, J. S., and W. J. Kraemer. Creatine supplementation: its effect on human muscular performance and body composition. J. Strength and Cond. Res. 10:200-210, 1996.

20. Wallace, M. et al. Effects of short-term creatine and sodium phosphate supplementation on body composition, performance and blood chemistry. Coaching and Sport Science J. 2:30-34, 1997.

21. MaArdle, W. D., Katch F. I., and Katch, V. L. Sports & Exercise, Nutritrion. 1999 Lippincott Williams & Wilkins 351 West Camden Street Baltimore, Md. USA

We claim:

1. A composition comprising:
a. creatine;
b. a phosphorus supplement, wherein the phosphorus supplement provides at least 75% of the recommended daily dose of phosphorus value per serving;
c. a blood buffer.

2. The composition according to claim 1, wherein the weight ratio of phosphorus to creatine is about 1:25 to about 10:1.

3. The composition according to claim 1, wherein the weight ratio of phosphorus to creatine is about 1:10 to about 1:1, preferably about 1:6 to about 1:4.

4. The composition according to claim 1, wherein the phosphorus supplement comprises an inorganic salt comprising phosphorus.

5. The composition according to claim 1, wherein the creatine is a creatine salt.

6. The composition according to claim 5, wherein the creatine is a highly hydrosoluble creatine salt.

7. The composition according to claim 5, wherein the creatine is an organic creatine salt.

8. The composition according to claim 7, wherein the creatine salt has a solubility above about 6 grams per 100 ml water.

9. The composition according to claim 7, wherein the creatine salt comprises an anionic component selected from the group of tartrate, maleate, malate, fumarate, citrate, and pyruvate.

10. The composition according to claim 1, wherein the blood buffer is selected from the group consisting of carbonate, bicarbonate, citrate and citric acid.

11. The composition according to claim 1, further comprising a Krebs cycle intermediate or precursor thereof.

12. The composition according to claim 11, wherein the anionic component of the creatine salt is a precursor of a Krebs cycle intermediate.

13. The composition according to claim 1, further comprising carbohydrate.

14. The composition according to claim 1, comprising 1-10 gram creatine, preferably provided by creatine citrate, 0.6-5 gram phosphorus, preferably provided by phosphate, 0.1-15 gram buffer, preferably a combination of carbonate and/or bicarbonate and citrate, and 1-100 g of digestible carbohydrates.

15. The composition according to claim 1, further comprising an effervescent.

16. The composition according to claim 1, further comprising a pentose, preferably ribose.

17. The composition according to claim 1, further comprising a sodium salt, preferably sodium phosphate.

18. A method for increasing the energy capacity within tissue cells comprising administrating to a subject a composition comprising creatine, a phosphorus supplement, wherein the phosphorus supplement provides at least 75% of the daily dose value per serving, and a blood buffer.

19. A method for increasing the anaerobic working capacity comprising, administrating to a subject a composition comprising creatine, a phosphorus supplement, wherein the phosphors supplement provides at least 75% of the daily dose value per serving, and a blood buffer.

20. A method for increasing the anaerobic working capacity wherein a subject is subjected to a building phase and subsequently to a maintenance phase, wherein said building phase comprises intake of a composition comprising creatine, a phosphorus supplement, wherein the phosphorus supplement provides at least 75% of the daily dose value per serving, and a blood buffer, and said maintenance phase comprises intake of said composition, wherein the intake quantity of the composition during maintenance phase is reduced by at least a factor 1.5.

21. A method according to claim 18, wherein the subject is human.

22. A method according to claim 21, wherein the subject is male.
